(19) United States
(12) Patent Application Publication
(10) Pub. No.: US 2023/0072783 A1
(43) Pub. Date: Mar. 9, 2023

(54) Medical Tubing Arrangement Comprising a Valve

(71) Applicant: Alexandria Tassopoulos, AURORA ONTARIO (CA)

(72) Inventor: Alexandria Tassopoulos, AURORA ONTARIO (CA)

(21) Appl. No.: 17/469,029

(22) Filed: Sep. 8, 2021

(51) Int. Cl. A61M 39/24; A61M 1/00

(52) U.S. Cl. CPC A61M 39/24; A61M 1/86; A61M 2039/244; A61M 2039/242

(57) ABSTRACT

A medical tubing arrangement comprises a length of tubing having at least a first end defining a first opening, and a check valve provided at the first end, spaced a predefined distance from the first opening, the check valve being of a resiliently deformable material and having at least one resiliently deformable flap, wherein the check valve is normally closed and, when closed, is configured to block flow of fluid out of the first opening. The medical tubing arrangement is configured to be connected to a suction apparatus by placing the first end over a suction port of the suction apparatus such that the at least one flap is configured to be urged inwardly by the suction port to open the check valve and permit flow of fluid through the valve and into the suction port, and removal of the suction port from the first end permits the at least one flap to close, thereby inhibiting flow of fluid through the check valve.

### FIELD OF DISCLOSURE

This disclosure relates to medical or surgical tubing and specifically to surgical tubing for connection to a suction apparatus. The disclosure provides for medical tubing incorporating a valve.

### BACKGROUND OF DISCLOSURE

The Inventor is familiar with the common surgical practice of using medical tubing during surgery to drain, and more particularly to suction, fluid from a patient. While tubing is used extensively in medicine to drain or convey fluid, the focus of this disclosure is medical tubing which is intended to be connected to a suction apparatus (e.g., a suction canister) which creates a negative pressure in the tubing to suck or draw fluid from a free end of the tubing, e.g., during surgery.

Medical tubing may be used to convey or suction various fluids emanating from a patient, e.g., blood, fat, lymph, pus, water, etc. Whatever the nature of the fluid, it is often undesirable, even contaminated, and should preferably remain contained. Accidents can occur, such as an end of medical tubing accidentally detaching from the suction canister. Even when intentionally disconnected, a pressure change could cause a squirt and general leaking is a problem. In addition to the suctioned fluids potentially being medically hazardous, they could create, e.g., a slippery floor which could result in a dangerous working environment.

Medical tubing is intended to be a disposable or sacrificial item. It is usually disposed of (or destroyed in accordance with medical waste procedures) after a single use. Accordingly, it needs to be relatively cheap. The Inventor is aware of an existing medical tubing assembly incorporating a movable ball-type valve (the ball may be of stainless steel). However, this assembly significantly increases the cost of the medical tubing and interferes with the disposability thereof.

The Inventor accordingly wishes to overcome the drawbacks noted above.

### SUMMARY OF DISCLOSURE

The disclosure provides a medical tubing arrangement comprising:
a length of tubing having at least a first end defining a first opening; and
a check valve provided at the first end, spaced a predefined distance from the first opening, the check valve being of a resiliently deformable material and having at least one resiliently deformable flap, wherein the check valve is normally closed and, when closed, is configured to block flow of fluid out of the first opening;
wherein the medical tubing arrangement is configured to be connected to a suction port of the suction apparatus by placing the first end over the suction port such that the at least one flap is configured to be urged inwardly by the suction port to open the check valve and permit flow of fluid through the valve and into the suction port, and removal of the suction port from the first end permits the at least one flap to close, thereby inhibiting flow of fluid through the check valve.

Medical tubing may be referred to as surgical tubing, suction tubing, medical tubes, etc. For the purposes of consistency, the term “medical tubing” will be used. The length of tubing may be, at least partially, of a resiliently deformable material.

The check valve may be of latex, silicon, rubber (natural or synthetic), polymer, a combination thereof, etc. The check valve may be of the same material as, or a different material from, the length of tubing.

“Normally closed” may mean that the check valve is biased, e.g., by means of the resiliently deformable material, towards a closed condition. The at least one flap may be resiliently biased towards a closed condition.

The length of tubing may include at least one head and a body of tubing, the head being provided at one end, or heads at both ends, of the body of tubing. The head may be configured for connection to the suction port. The head may be at the first end and define the first opening.

The check valve may be formed or manufactured integrally with the length of tubing; in other words, the check valve and length of tubing may be of monolithic construction. Instead, the check valve may be manufactured separately from the length of tubing and then embedded or inserted therein or connected thereto. The length of tubing may include a seat at the first end to accommodate the check valve. The check valve may be integrated into a head which is then coupled to a body of tubing to comprise the length of tubing.

The check valve may be a cross-slit valve or a duckbill valve. If the valve is a cross-slit valve, it may have one or more of the following characteristics:
three, four, or more flaps;
the flaps may be transverse to a length direction of the length of tubing in their closed condition; and/or
the flaps may be inclined or parallel to the length direction of the length of tubing in their open condition.

If the valve is a duckbill valve, it may have one or more of the following characteristics:
one or two flaps;
the flaps may be inclined relative to the length direction of the length of tubing in their closed condition; and/or
the flaps may be more inclined or parallel to the length direction of the length of tubing in their open condition.

The length of tubing may include a second end having a second opening. The second end may be conventional (that is, the same as an end of conventional medical tubing). The second end may be used for suctioning fluid from a patient. The first end may thus be a canister end and the second end may be a patient end.

In one embodiment, the medical tubing arrangement may have the check valve at the first end only. The medical tubing arrangement may thus be directional, with a flow direction being along the length of tubing and out of the first opening. The medical tubing arrangement may include indicia to indicate which end is the first end.

In another embodiment, the medical tubing arrangement may include identical check valves at each end, the medical tubing arrangement thus being bidirectional or reversable.

The suction port may be connected to, or form part of, a conventional suction canister or Fluid Waste Management System (FWMS). The suction port is typically shaped as a cylindrical spigot (which may be slightly tapered) and is about 1 inch (about 2-3 cm) in length. The predefined distance that the check valve is spaced from the first opening may thus be less than the length of the suction port. The predefined distance may be 0-1 inches (about 0-3 cm) and may be about 0.5 inches (about 1-2 cm). This may ensure that the suction port penetrates the check valve to displace the at least one flap inwardly, thereby opening the check valve.

The disclosure extends to a tubing head incorporating a check valve, the tubing head being for attachment to a tubing body thereby to comprise the medical tubing arrangement defined above.

The disclosure extends to a method of connecting the medical tubing arrangement as defined above to the suction port, the method comprising placing the first end over the suction port, thereby causing the suction port to be inserted into the first opening and urging the at least one flap to open.

The method may further comprise removing the first end from the suction port causing the suction port to be withdrawn from the first opening, thereby permitting the at least one flap to close. The closing may be automatic due to the resilient character of the at least one flap.

### BRIEF DESCRIPTION OF DRAWINGS

The disclosure will now be further described, by way of example, with reference to the accompanying diagrammatic drawings.

In the drawings:

FIG. 1 shows a three-dimensional view of a medical tubing arrangement, in accordance with a first example embodiment of the disclosure;

FIG. 2 shows an exploded view of the medical tubing arrangement of FIG. 1;

FIG. 3 shows an axial-sectional view of the medical tubing arrangement of FIG. 1;

FIG. 4 shows an enlarged view of a check valve, in a closed condition, of the medical tubing arrangement of FIG. 1;

FIG. 5 shows an enlarged view of a check valve, in an open condition, of the medical tubing arrangement of FIG. 1;

FIG. 6 shows an axial-sectional view of the medical tubing arrangement of FIG. 1, being connected to a suction port;

FIG. 7 shows an axial-sectional view of the medical tubing arrangement of FIG. 6, fully connected to the suction port;

FIG. 8 shows an axial-sectional view of the medical tubing arrangement of FIG. 7, being disconnected from the suction port;

FIG. 9 shows a three-dimensional view of a medical tubing arrangement, in accordance with a second example embodiment of the disclosure;

FIG. 10 shows an exploded view of the medical tubing arrangement of FIG. 9;

FIG. 11 shows an axial-sectional view of the medical tubing arrangement of FIG. 9;

FIG. 12 shows an enlarged view of a check valve, in a closed condition, of the medical tubing arrangement of FIG. 9;

FIG. 13 shows an enlarged view of a check valve, in an open condition, of the medical tubing arrangement of FIG. 9;

FIG. 14 shows an axial-sectional view of the medical tubing arrangement of FIG. 9, being connected to the suction port;

FIG. 15 shows an axial-sectional view of the medical tubing arrangement of FIG. 14, fully connected to the suction port; and

FIG. 16 shows an axial-sectional view of the medical tubing arrangement of FIG. 15, being disconnected from the suction port.

### DETAILED DESCRIPTION OF EXAMPLE EMBODIMENT

The following description of an example embodiment of the disclosure is provided as an enabling teaching of the disclosure. Those skilled in the relevant art will recognize that changes can be made to the example embodiment described, while still attaining the beneficial results of the present disclosure. It will also be apparent that some of the desired benefits of the present disclosure can be attained by selecting some of the features of the example embodiment without utilizing other features. Accordingly, those skilled in the art will recognize that modifications and adaptations to the example embodiment are possible and can even be desirable in certain circumstances and are a part of the present disclosure. Thus, the following description of the example embodiment is provided as illustrative of the principles of the present disclosure and not a limitation thereof.

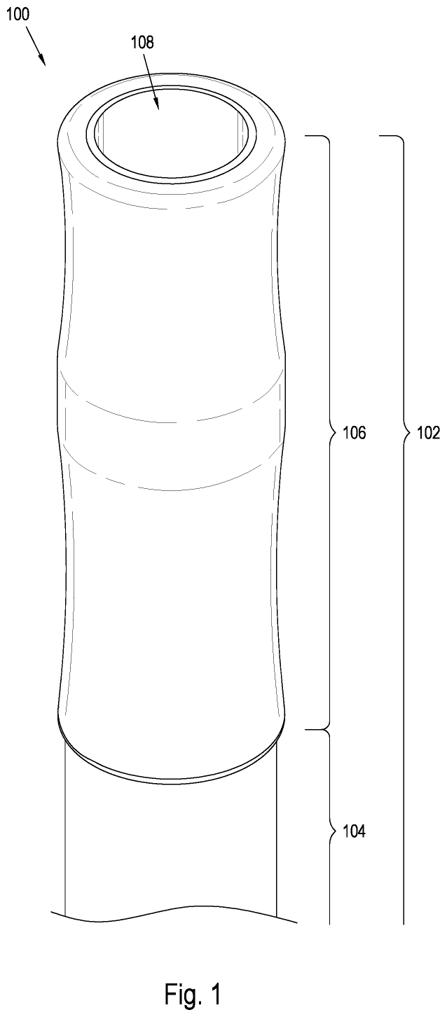

FIG. 1 illustrates a medical tubing arrangement 100 in accordance with a first example embodiment. The medical tubing arrangement 100 comprises a length of tubing 102 which in turn comprises a body of tubing 104 and a tubing head 106. The length of tubing 102 is intended for medical (especially surgical) applications by connecting it to the suction port of a Fluid Waste Management System (FWMS)—more on this below.

The body of tubing 104 and the head 106 may be manufactured separately and joined (e.g., bonded or adhered) together. The length of tubing 102 is of indeterminate length and made, at least partially, of a resiliently deformable material, e.g., medical grade silicon. FIG. 1 illustrates a first end of the length of tubing 102 which defines a first opening 108.

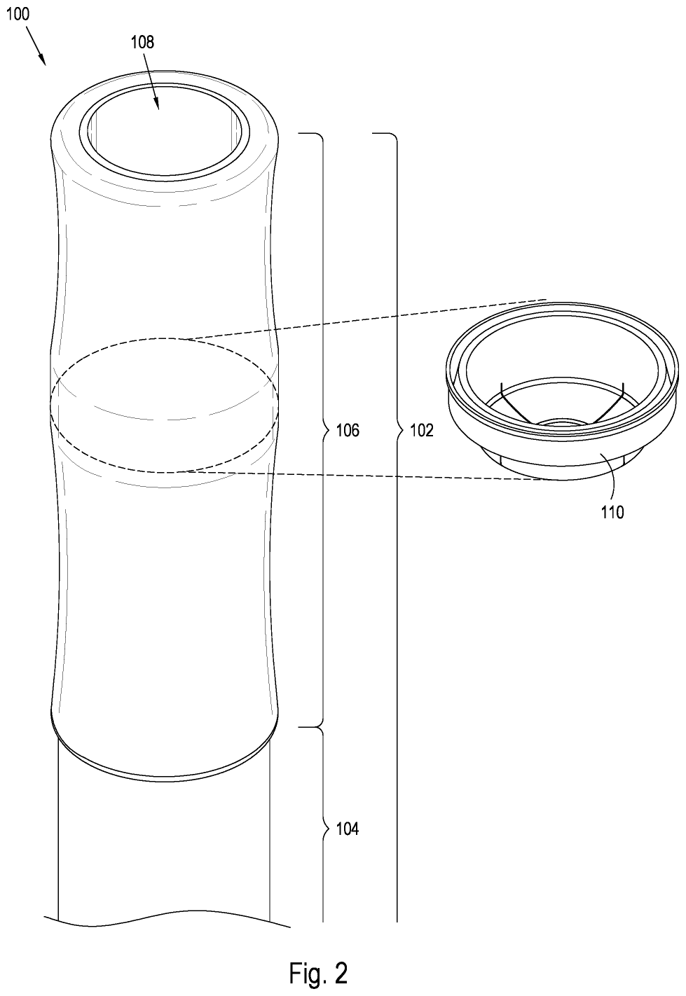

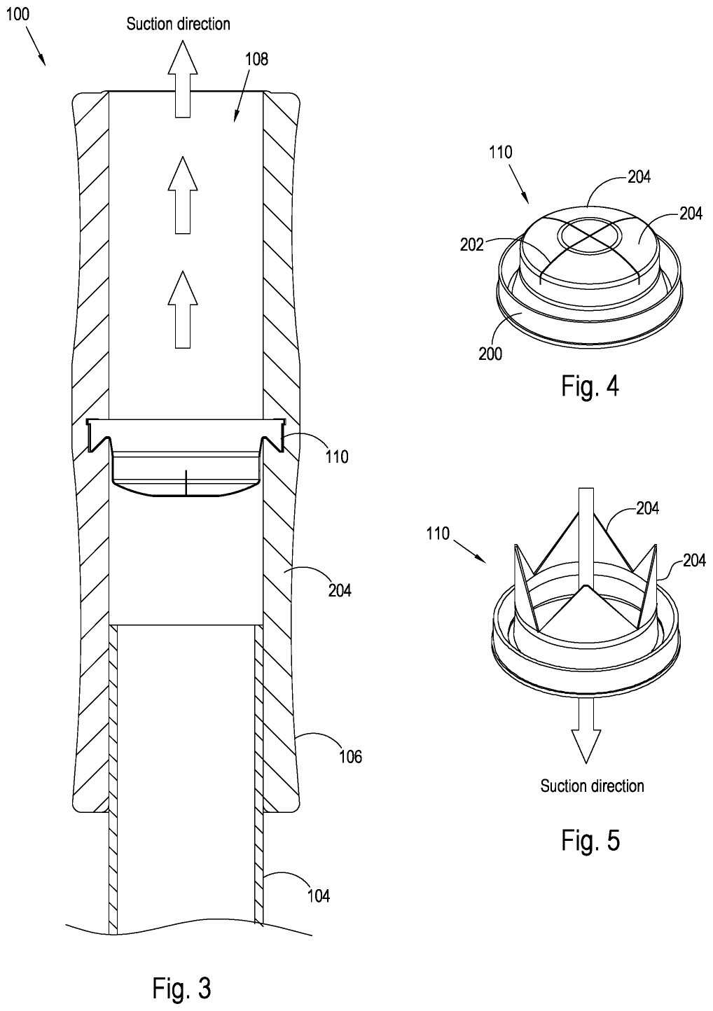

FIGS. 2-3 illustrate an important feature of the present disclosure; the medical tubing arrangement 100 comprises a valve, specifically a check valve 110, provided at the first end. The check valve is spaced a short distance, e.g., 0.5 inches, inwardly of the first opening. The check valve 110 is of a resiliently deformable material, e.g., silicon or a polymer.

Various configurations of the check valve 110 may be practicable and FIGS. 2-9 illustrate a first example embodiment in the form of a cross-slit check valve 110. As such, the cross-slit check valve 110 has a central slit (in the form of a +) 202 which defines four resiliently deformable flaps 204, each being about 90°. The check valve 110 is configured to be normally closed; in other words, the flaps 204 are biased to be transverse to, and block off, a flow direction of the length of tubing 102.

The check valve 110 (shown most clearly in FIGS. 4-5) has a peripherally extending collar or lip 200 which is configured to be embedded in a sidewall of the tubing head 106. Thus, the check valve 110 is non-removably integrated with the tubing head 106 which, in turn, is fixed to the tubing body 104, thereby creating an integrated medical tubing assembly 100. This connection and integration may be part of the manufacturing process and the components (the tubing body 104, the tubing head 106, and the check valve 110) may be non-separable and thus usable and disposable as a single article.

An intended direction of flow is illustrated in FIG. 3: out of the first opening 108. This is because this is the suction direction provided by the FWMS; a negative pressure is applied to the first opening which thereby creates the suction and dictates the flow direction. In addition to being normally closed, the check valve 110 is configured to block or inhibit fluid flow in the suction direction. In fact, application of a suction may serve to reinforce the biasing action of the flaps 204 and close the check valve 110 even more tightly.

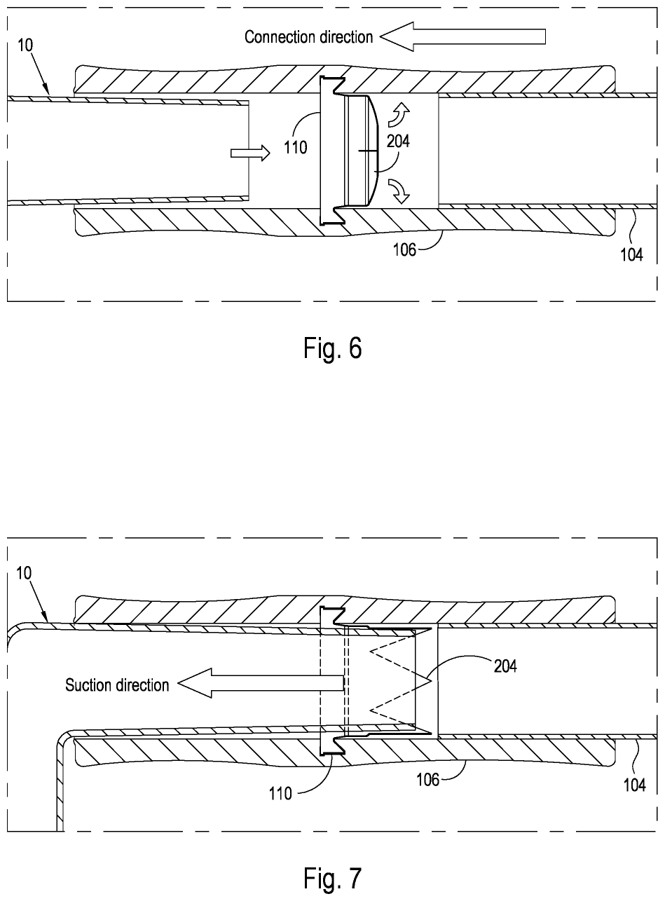

FIGS. 6-8 illustrate operation of the medical tubing assembly 100. The only part of the FWMS which is illustrated is a suction port 10. The FWMS and the suction port 10 are entirely conventional and need not be modified to work in accordance with the medical tubing assembly 100. In accordance with prior art techniques, the suction port 10 is connected or plumbed to a fluid storage reservoir and has, in use, a negative pressure provided thereto.

The tubing head 106 is connected over the suction port 10 as illustrated in FIG. 6 (stated conversely, the suction port 10 is inserted into the first opening 108 of the tubing head 106). As the tubing head 106 is pushed further over the suction port 10, an end of the suction port 10 begins to bear against the check valve 110 and force the flaps 204 inwardly, thereby to open against their bias.

When fully connected, the suction port 10 projects through the peripheral collar 200 of the check valve 110 to force the flaps 204 into a fully open condition. The flaps 204 are forced, by the physical presence of the suction port 10, inwards/backwards and against an inner wall of the tubing head 106. The check valve 110 is thus forcibly opened by the action of coupling the medical tubing assembly 100 to the suction port 10 of the FWMS.

For this reason, the check valve 110 should be spaced a distance from the first opening 108 less than a length of the suction port 10. The suction port 10 could, although it need not necessarily, project all the way past the flaps 204. In FIG. 7, the suction port 10 stops just short of pushed-back tips of the flaps 204. A smallest diameter of the connected medical tubing assembly 100 is thus defined by the suction port 10 and not by the check valve 110; accordingly, the presence of the check valve 110 may not provide flow limitation or obstruction compared to the suction port 10 (which is used in prior art systems anyway).

The medical tubing assembly 100 may then be used by a medical professional, e.g., a medical assistant applying a second end of the medical tubing assembly 100 (not illustrated) to a wound or operation area of a patient to remove fluid by suctioning. In fact, the medical professional may be entirely oblivious to the existence or operation of the check valve 110. In other words, the presence of the check valve 100 need not change the way the medical professionals operate.

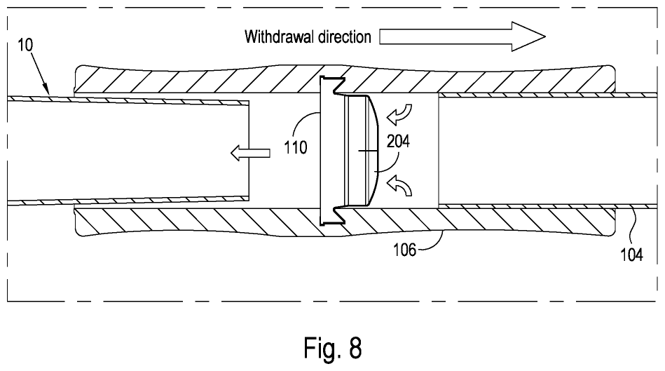

When the medical tubing assembly 100 is disconnected from the suction port 10, the suction port 10 is merely withdrawn (see FIG. 8) from the first opening 108 of the tubing head 106. The removal of the suction port 10 from inside the check valve 110 permits the flaps 204 to close under their own bias. Further, if any suction force remains from the first opening 108, this urges the flaps 204 to close even more quickly.

Accordingly, any fluid (not illustrated) in the length of tubing 102 (at least, behind the check valve 110) will be prevented or inhibited from exiting the medical tubing assembly 100. This may prevent or reduce leaks of fluid from the medical tubing assembly 100. Such leaks can occur when tubing (in general) is removed from the suction port 10, the removal either being intentional or inadvertent.

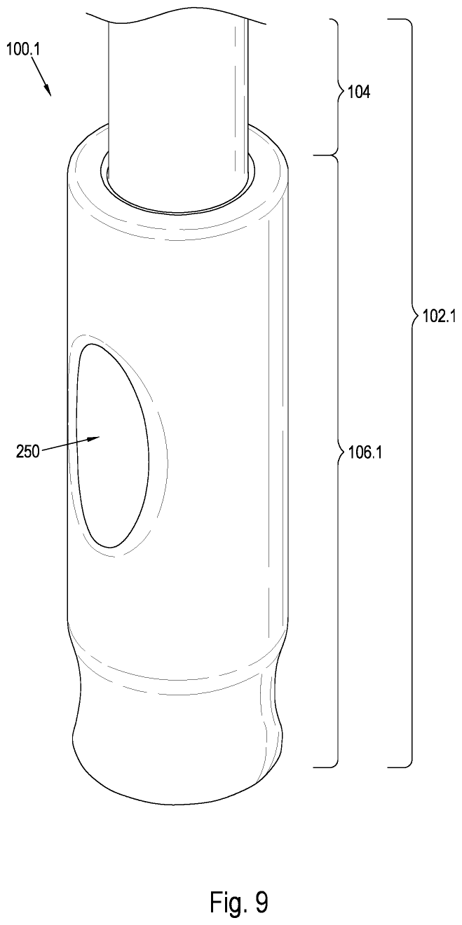

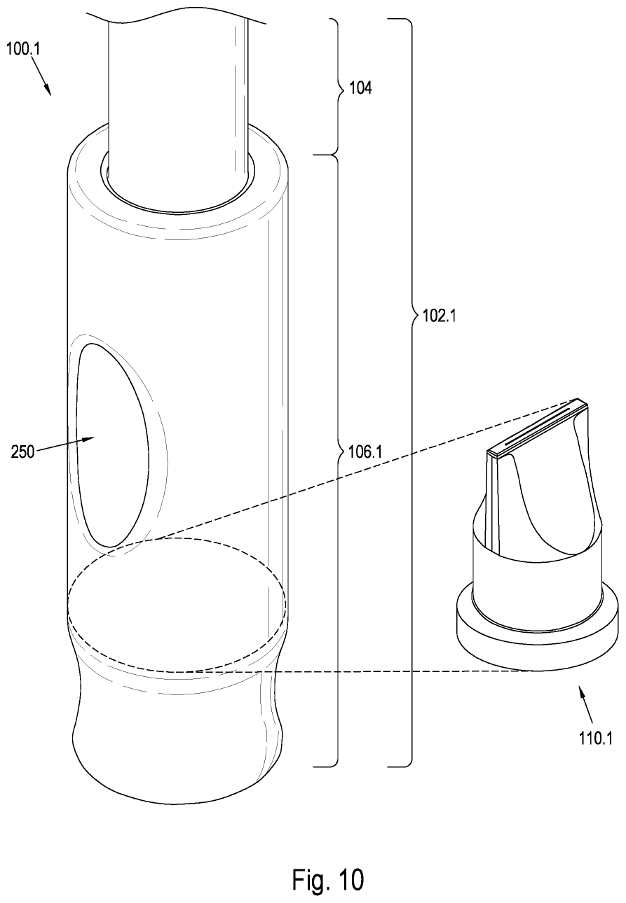

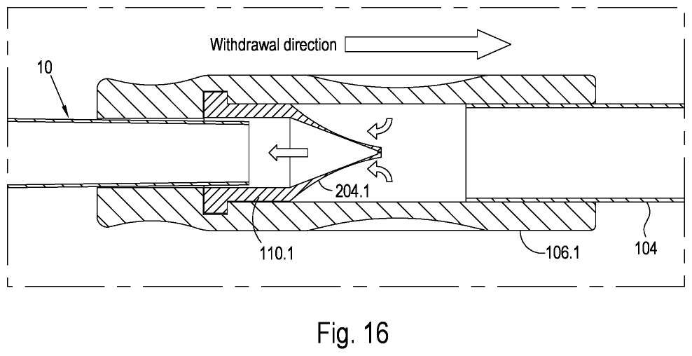

FIGS. 9-16 illustrate a second example embodiment of a medical tubing arrangement 100.1 in accordance with the present disclosure. FIGS. 9-16 correspond to FIGS. 1-8 and the same or similar numerals refer to the same or similar features. In principle, the medical tubing arrangement 100.1 functions the same as the medical tubing arrangement 100, so only the differences will be discussed, many of which may be cosmetic or alternative design choices.

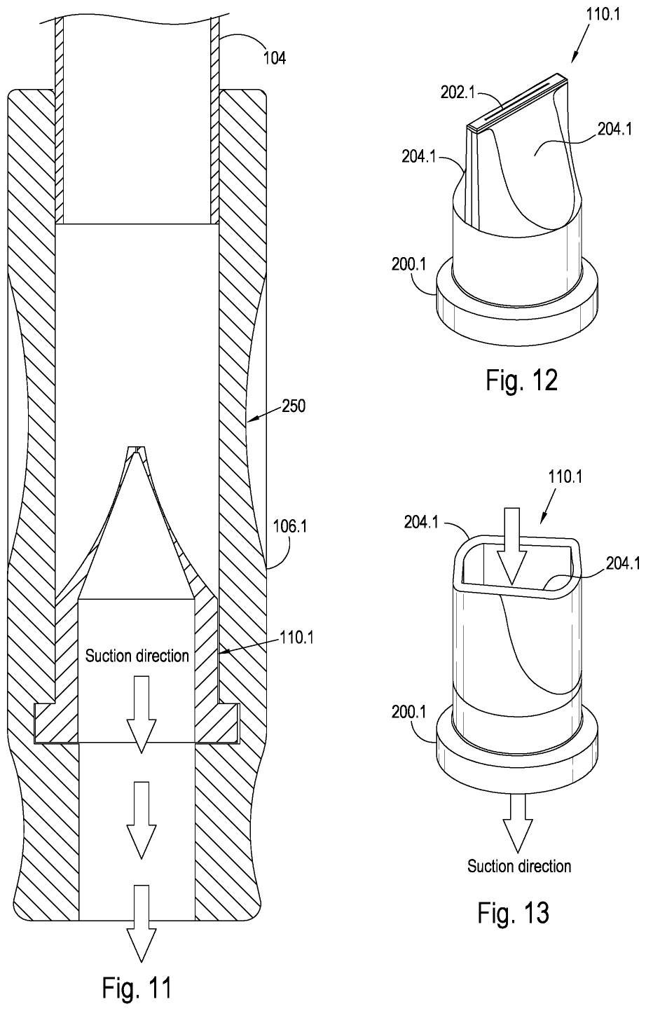

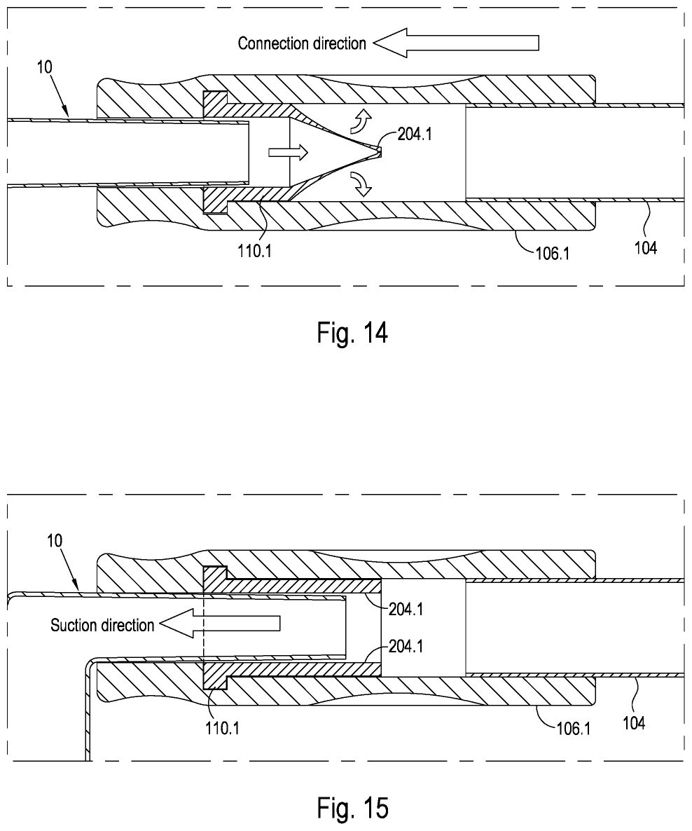

Instead of a cross-slit check valve 110, a duckbill check valve 110.1 is provided inside the tubing head 106.1 of the medical tubing arrangement 100.1. The duckbill check valve 110.1 has a single, central slit 202.1 and a pair of flaps 204.1 either side of the slit 202.1. If the flaps 204 of the cross-slit check valve 110 were flat (that is, inclined at 0°) in their closed condition, then the flaps 204.1 of the duckbill check valve 110.1 are inclined at 45°-75° (see FIGS. 11-12). When forced open by the suction port 10, the flaps 204.1 are forced towards an inner sidewall of the tubing head 106.1 (see FIG. 15). The operation is otherwise the same, illustrated in FIGS. 14-16.

It may be desirable to have the flaps 204, 204.1 pressed as closely, or as flush, to an inner wall of the length of tubing 102, 102.1 as possible. This may prevent build-up of fluid or clogging of particulate matter carried in the fluid. To this end, the flaps 204, 204.1 may be configured to be pressed or urged all the way back, that is, against and flush with the length of tubing 102, 102.1. Also, the spacing of the check valve 110, 110.1 may be sufficiently short from the first opening 108 such that the suction port 10, when coupled, projects all the way through the check valve 110, 110.1, thus effectively sandwiching the flaps 204, 204.1 between an outer wall of the suction port 10 and the inner wall of the length of tubing 102, 102.1.

Another difference in the second example embodiment of the medical tubing arrangement 100.1 is the addition of gripping indentations 250 in an outer surface of the tubing head 106.1. The gripping indentations 250 may assist a user in grasping the tubing head 106.1 and coupling it to the suction port 10, to assist or ensure that the medical tubing arrangement 100.1 is fully coupled to the suction port 10, in other words, that the suction port 10 has fully opened the flaps 204.1. Other indicia could be provided on the tubing head 106, 106.1 or elsewhere on the medical tubing arrangement 100, 100.1, e.g., warnings or directions like “Check valve inside” or “Ensure fully coupled” or merely an arrow or pictogram.

The Applicant believes that the disclosure, as exemplified, may have the following advantages:
The valve 110, 100.1 is automatically opened simply by coupling the medical tubing arrangement 100, 100.1 to the suction port 10.
The valve 110, 100.1 is automatically closed simply by removing the medical tubing arrangement 100, 100.1 to the suction port 10.
This coupling and removing, from the user's perspective, is similar or identical to the usage of prior art medical tubing. Accordingly, the operation of the check valve 110, 110.1 is user-agnostic.
Leakage of fluids, especially medical- or patient-sourced fluids, is potentially dangerous. They could constitute a biohazard or create an unsafe working environment, e.g., by making floors slippery.
The medical tubing arrangement 100, 100.1 is relatively cheap. The FWMS is far more expensive and needs no modification for usage with the medical tubing arrangement 100, 100.1.
The check valve 110, 110.1 does not inhibit flow or cause blockages as the inner diameter of the suction port 10 is still the limiting factor from a flow perspective.
While the inclusion of the check valve 110, 110.1 may slightly increase the cost of the medical tubing arrangement 100, 100.1, it is still comparatively cheap and the medical tubing arrangement 100, 100.1 may therefore continue to be a disposable or sacrificial component.
The prior disclosure of which the Applicant is aware, of a movable ball-type valve, may not work well for surgical procedures because it would not allow fluid to flow freely and may clog due to particulate matter like blood clots, tissue, and/or bone debris, etc. In contrast, the present disclosure does not present constrictions smaller than the diameter of the suction port and does not present crevices or obstructions where such particulate matter may get snagged/lodged. As the flaps 204, 204.1 are folded and urged flush against and inner wall of the length of tubing 102, fluid flow is unrestricted.

What is claimed is:

1. A medical tubing arrangement comprising:
a length of tubing having at least a first end defining a first opening; and
a check valve provided at the first end, spaced a predefined distance from the first opening, the check valve being of a resiliently deformable material and having at least one resiliently deformable flap, wherein the check valve is normally closed and, when closed, is configured to block flow of fluid out of the first opening;
wherein the medical tubing arrangement is configured to be connected to a suction apparatus by placing the first end over a suction port of the suction apparatus such that the at least one flap is configured to be urged inwardly by the suction port to open the check valve and permit flow of fluid through the valve and into the suction port, and removal of the suction port from the first end permits the at least one flap to close, thereby inhibiting flow of fluid through the check valve.

2. The medical tubing arrangement of claim 1, wherein the check valve is biased towards a closed condition.

3. The medical tubing arrangement of claim 2, wherein the at least one flap is resiliently biased towards the closed condition.

4. The medical tubing arrangement of claim 1, wherein the length of tubing includes at least one head and a body of tubing, the head being provided at one end, or heads at both ends, of the body of tubing.

5. The medical tubing arrangement of claim 1, wherein the head is at the first end and defines the first opening and is configured for connection to the suction port.

6. The medical tubing arrangement of claim 1, wherein:
the check valve is formed or manufactured integrally with the length of tubing; or
the check valve is manufactured separately from the length of tubing and then embedded or inserted therein or connected thereto.

7. The medical tubing arrangement of claim 1, wherein the check valve is a cross-slit valve or a duckbill valve.

8. The medical tubing arrangement of claim 7, wherein the valve is a cross-slit valve and has one or more of the following characteristics:
three, four, or more flaps;
the flaps may be transverse to a length direction of the length of tubing in their closed condition; and/or
the flaps may be inclined or parallel to the length direction of the length of tubing in their open condition.

9. The medical tubing arrangement of claim 7, wherein the valve is a duckbill valve and has one or more of the following characteristics:
one or two flaps;
the flaps may be inclined relative to a length direction of the length of tubing in their closed condition; and/or
the flaps may be more inclined or parallel to the length direction of the length of tubing in their open condition.

10. The medical tubing arrangement of claim 1, wherein the first end is configured for connection to the suction port forming part of a conventional suction canister or Fluid Waste Management System (FWMS).

11. The medical tubing arrangement of claim 1, wherein the predefined distance that the check valve is spaced from the first opening is less than a length of the suction port.

12. The medical tubing arrangement of claim 11, wherein the predefined distance is 0-1 inches (about 0-3 cm), thereby to ensure that the suction port penetrates the check valve to displace the at least one flap inwardly, thereby opening the check valve.

13. The medical tubing arrangement of claim 1, wherein the at least one flap of the check valve is configured to be urged fully inwardly by the suction port such that the at least one flap is flat against, or flush with, an inner wall of the length of tubing.

14. A tubing head incorporating a check valve, the tubing head being for attachment to a tubing body thereby to comprise the medical tubing arrangement of claim 4.

15. A method of connecting the medical tubing arrangement of claim 1 to the suction port, the method comprising placing the first end over the suction port, thereby causing the suction port to be inserted into the first opening and to urge the at least one flap inwardly.

16. The method of claim 15, further comprising removing the first end from the suction port causing the suction port to be withdrawn from the first opening, thereby permitting the at least one flap to close under its bias.
